(19) United States
(12) Patent Application Publication
(10) Pub. No.: US 2005/0100527 A1
(43) Pub. Date: May 12, 2005

(54) Compounds for delivering substances into cells

(76) Inventors: Yong Liang Chu, Rockville, MD (US); Frank Q. Li, Montgomery Village, MD (US); Jian-Tai Qiu, Rockville, MD (US); Jerry Lin, Bethesda, MD (US)

Correspondence Address: Supervisor;Patent Prosecution Services, PIPER RUDNICK LLP, 1200 Nineteenth Street, N.W., Washington, DC, 20036-2412, US

(21) Appl. No.: 11/007,267

(22) Filed: Dec. 9, 2004

Related U.S. Application Data
(62) Division of application No. 10/035,223, filed on Jan. 4, 2002.

(51) Int. Cl. A61K 31/785; C08F 283/00

(52) U.S. Cl. 424/78.27; 525/540

(57) ABSTRACT

Lipids and compositions of lipids that can be used as lipid aggregates (i.e., liposomes) for the delivery of macromolecules and other compounds into cells are provided. The lipids have a general structure represented by the formula: 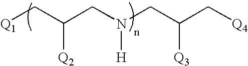 The lipids can be used to form lipid aggregates (i.e., liposomes). These lipid aggregates can serve as transfection reagents for the delivery of various compounds into cells. Suitable compounds that can be delivered into cells include nucleic acids (e.g. DNA, RNA), oligonucleotides, proteins, peptides, and small molecular drugs.

### BACKGROUND OF THE INVENTION

1. Field of the Invention

The present invention relates to lipid compounds that can be used in lipid aggregates (i.e., liposomes) for the delivery of macromolecules and other substances into cells.

2. Background of the Technology

Various methodologies have been used to transfect macromolecules such as DNA, including microinjection, protoplast fusion, liposome fusion, calcium phosphate precipitation, electroporation and retroviruses. All of these methods suffer from some significant drawbacks: they tend to be too inefficient, too toxic, too complicated or too tedious to be conveniently and effectively adapted to biological and/or therapeutic protocols on a large scale. For instance, the calcium phosphate precipitation method can successfully transfect only about 1 in 10⁷ to 1 in 10⁴ cells. This frequency is too low to be applied to current biological and/or therapeutic protocols. Microinjection is efficient but not practical for large numbers of cells or for large numbers of patients. Protoplast fusion is more efficient than the calcium phosphate method but the propylene glycol that is required is toxic to the cells. Electroporation is more efficient than calcium phosphate but requires a special apparatus. Retroviruses are sufficiently efficient but the introduction of viruses into the patient leads to concerns about infection and cancer.

Lipid aggregates (e.g., liposomes) have also been found to be useful as agents for delivery to introduce macromolecules, such as DNA, RNA, protein, and small chemical compounds such as pharmaceuticals, into cells. In particular, lipid aggregates comprising cationic lipid components have been shown to be especially effective for delivering anionic molecules into cells. In part, the effectiveness of cationic lipids is thought to result from enhanced affinity for cells, many of which bear a net negative charge. Additionally, the net positive charge on lipid aggregates comprising a cationic lipid enables the aggregate to bind polyanions, such as nucleic acids. Lipid aggregates containing DNA are known to be effective agents for efficient transfection of target cells.

Liposomes are microscopic vesicles consisting of concentric lipid bilayers. The lipid bilayers of liposomes are generally organized as closed concentric lamellae, with an aqueous layer separating each lamella from its neighbor. Vesicle size typically falls in a range of between about 20 and about 30,000 nm in diameter. The liquid film between lamellae is usually between about 3 and 10 nm thick.

The structure of various types of lipid aggregates varies, depending on composition and method of forming the aggregate. Such aggregates include liposomes, unilamellar vesicles (ULVs), multilameller vesicles (MLVs), micelles and the like, having particular sizes in the nanometer to micrometer range. Methods of making lipid aggregates are by now well-known in the art. The main drawback to use of conventional phospholipid containing liposomes for delivery is that the material to be delivered must be encapsulated and the liposome composition has a net negative charge which is not attracted to the negatively charged cell surface. By combining cationic lipid compounds with a phospholipid, positively charged vesicles and other types of lipid aggregates can bind DNA, which is negatively charged, and can be taken up by and can transfect target cells. See, for example, Felgner et al., Proc. Natl. Acad. Sci. USA 84, 7413-7417 (1987); U.S. Pat. Nos. 4,897,355 and 5,171,678 and International Publication No. WO 00/27795.

Liposomes may be prepared by a number of methods. Preparing MLV liposomes usually involves dissolving the lipids in an appropriate organic solvent and then removing the solvent under a gas or air stream. This leaves behind a thin film of dry lipid on the surface of the container. An aqueous solution is then introduced into the container with shaking in order to free lipid material from the sides of the container. This process disperses the lipid, causing it to form into lipid aggregates or liposomes. LUV liposomes may be made by slow hydration of a thin layer of lipid with distilled water or an aqueous solution of some sort.

Liposomes may also be prepared by lyophilization. This process comprises drying a solution of lipids to a film under a stream of nitrogen. This film is then dissolved in a volatile solvent, frozen, and placed on a lyophilization apparatus to remove the solvent. To prepare a pharmaceutical formulation containing a drug or other substance, a solution of the substance is added to the lyophilized lipids, whereupon liposomes are formed.

A variety of methods for preparing various liposomes have been described in the periodical and patent literature. For specific reviews and information on liposome formulations, reference is made to reviews by Pagano et al., Ann. Rev. Biophysic. Bioeng., 7, 435-68 (1978) and Szoka et al., Ann. Rev. Biophysic. Bioeng., 9, 467-508 (1980) and U.S. Pat. Nos. 4,229,360; 4,224,179;.4,241,046; 4,078,052; and 4,235,871.

Various biological substances have been encapsulated into liposomes by contacting a lipid with the matter to be encapsulated and then forming the liposomes as described above. A drawback of these methods is that the fraction of material encapsulated into the liposome structure is generally less than 50%, usually less than 20%, often necessitating an extra step to remove unencapsulated material. An additional problem, related to the above, is that after removal of unencapsulated material, the encapsulated material can leak out of the liposome. This second issue represents a substantial stability problem to which much attention has been addressed in the art.

Despite advances in the field, a need remains for a variety of improved lipid compounds. Since different cell types differ from one another in membrane composition, different compositions and types of lipid aggregates have been found to be effective for different cell types, either for their ability to contact and fuse with target cell membranes, or for aspects of the transfer process itself. At present these processes are not well understood, consequently the design of effective liposomal precursors is largely empirical. Besides content and transfer, other factors are of importance include the ability to form lipid aggregates suited to the intended purpose, the possibility of transfecting cells in the presence of serum, toxicity to the target cell, stability as a carrier for the compound to be delivered, and ability to function in an in vivo environment. In addition, lipid aggregates can be improved by broadening the range of substances which can be delivered into cells.

The lipid compounds of the present invention have improved function with respect to several of the foregoing attributes.

### SUMMARY OF THE INVENTION

A compound having a general structure represented by the formula: 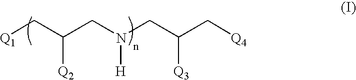
is provided wherein:

n is 0 or a positive integer;

Q₁ is N(R)₃+, N(R)₂, O(R), or O(R)₂+ wherein each R substituent is independently selected from the group consisting of H, a straight chain or branched alkyl or alkenyl, a straight chain or branched alkyl or alkenyl ether, a straight chain or branched alkyl or alkenyl ester and a straight chain or branched alkyl or alkenyl carbonyldioxide with the proviso that at least one R substituent on the O or N atom of Q₁ is not H;

Q₃, and each Q₂ are independently selected from the group consisting of H, O(R′), N(R′)₂, NH(R″), and S(R′); and

Q₄ is selected from the group consisting of N(R′)₂, and NH(R″); wherein:

R′ is H or one the following moieties:

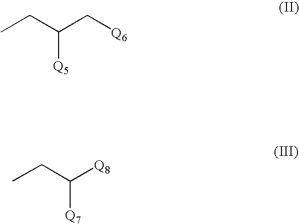

and wherein each of Q₅, Q₆, Q₇ and Q₈ are independently selected from the group consisting of N(R)₃+, N(R)₂, OR, O(R)₂+, O(R′), N(R′)₂, NH(R″), S(R), S(R)₂+ and S(R′); wherein each R substituent on Q₅, Q₆, Q₇ or Q₈ is independently selected from H or a methyl group;

each R′ substituent on Q₅, Q₆, Q₇ or Q₈ is as defined above for Q₄; and

each R″ substituent on Q₂, Q₃, Q₄, Q₅, Q₆, Q₇ or Q₈ is independently hydrogen or comprises a moiety selected from the group consisting of amino acid residues, polypeptide residues, protein residues, carbohydrate residues and combinations thereof.

According to a preferred embodiment of the invention, the compound comprises a total of at least two R′ substituents on each N, O or S atom of Q₂, Q₃ and/or Q₄ which are represented by formula II or formula III.

A kit comprising a compound as set forth above in formula I and at least one additional component is also provided. The additional component may be one or more cells, a cell culture media, a nucleic acid, or a transfection enhancer.

A method for introducing a substance into cells is also provided. The method comprises forming a liposome from a compound as set forth above, contacting the liposome with the substance to form a complex between the liposome and the substance and incubating the complex with one or more cells. The substance may be a nucleic acid or a biologically active substance.

### BRIEF DESCRIPTION OF THE DRAWINGS

The present invention may be better understood with reference to the accompanying drawings in which:

FIG. 1A shows a method of synthesizing a lipid precursor having alkyl substituents according to a first embodiment of the invention;

FIG. 1B shows a method of synthesizing a lipid precursor having alkenyl substituents according to another embodiment of the invention;

FIGS. 2A-2E show methods of synthesizing lipid compounds according to the invention from the lipid precursor of FIG. 1;

FIG. 3 shows a method of synthesizing a lipid precursor according to a second embodiment of the invention from an intermediate product of the synthesis depicted in FIG. 1;

FIGS. 4A-4C show methods of synthesizing lipid compounds according to the invention from the lipid precursor of FIG. 3;

FIG. 5 shows a method of synthesizing a lipid precursor according to a further embodiment of the invention;

FIGS. 6A and 6B show methods of synthesizing lipid compounds according to the invention from the lipid precursor of FIG. 5;

FIG. 7A shows a method of synthesizing a lipid precursor according to the invention from an intermediate product of the synthesis depicted in FIG. 1;

FIG. 7B shows a method of synthesizing a lipid precursor according to the invention from an intermediate product of the synthesis depicted in FIG. 7A;

FIG. 7C shows a method of synthesizing a different lipid precursor according to the invention from an intermediate product of the synthesis depicted in FIG. 7A;

FIG. 8 shows a method of synthesizing a lipid compound according to the invention from the lipid precursor of FIG. 1;

FIGS. 9A-9G show various lipid compounds according to another embodiment of the invention;

FIGS. 10A-10C show various lipid compounds according to a further embodiment of the invention; and

FIG. 11 shows another embodiment of a lipid compound according to the invention.

### DETAILED DESCRIPTION OF THE INVENTION

The present invention relates to cationic lipids and compositions of cationic lipids having utility in lipid aggregates for delivery of macromolecules and other compounds into cells. Lipids according to a first embodiment of the invention have a general structure represented by formula I below: 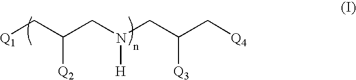
wherein:

n is 0 or a positive integer;

Q₁ is N(R)₃+, N(R)₂, O(R), or O(R)₂+ wherein each R substituent is independently selected from the group consisting of H, a straight chain or branched alkyl, a straight chain or branched alkyl ether, a straight chain or branched alkyl ester and a straight chain or branched alkyl carbonyldioxide with the proviso that at least one R substituent on the O or N atom of Q₁ is not H;

Q₃, and each Q₂ are independently selected from the group consisting of H, O(R′), N(R′)₂, NH(R″), and S(R′); and

Q₄ is selected from the group consisting of N(R′)₂, and NH(R″); wherein:

R′ is H or one the following moieties:

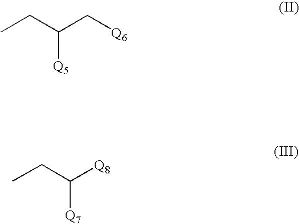

and wherein each of Q₅, Q₆, Q₇ and Q₈ are independently selected from the group consisting of N(R)₃+, N(R)₂, OR, O(R)₂+, O(R′), N(R′)₂, NH(R″), S(R), S(R)₂+ and S(R′); wherein each R substituent on Q₅, Q₆, Q₇ or Q₈ is independently selected from H or a methyl group;

each R′ substituent on Q₅, Q₆, Q₇ or Q₈ is as defined above for Q₄; and

each R″ substituent on Q₂, Q₃, Q₄, Q₅, Q₆, Q₇ or Q₈ is independently hydrogen or comprises a moiety selected from the group consisting of amino acid residues, polypeptide residues, protein residues, carbohydrate residues and combinations thereof.

According to a preferred embodiment of the invention, the compound comprises a total of at least two R′ substituents on each N, O or S atom of Q₂, Q₃ and/or Q₄ which are represented by formula II or formula III. Therefore, when n=0 and Q₃ is H, Q₄ is preferably N(R′)₂ and both R′ substituents on the Q₄ nitrogen atom are preferably represented by formula II or formula III.

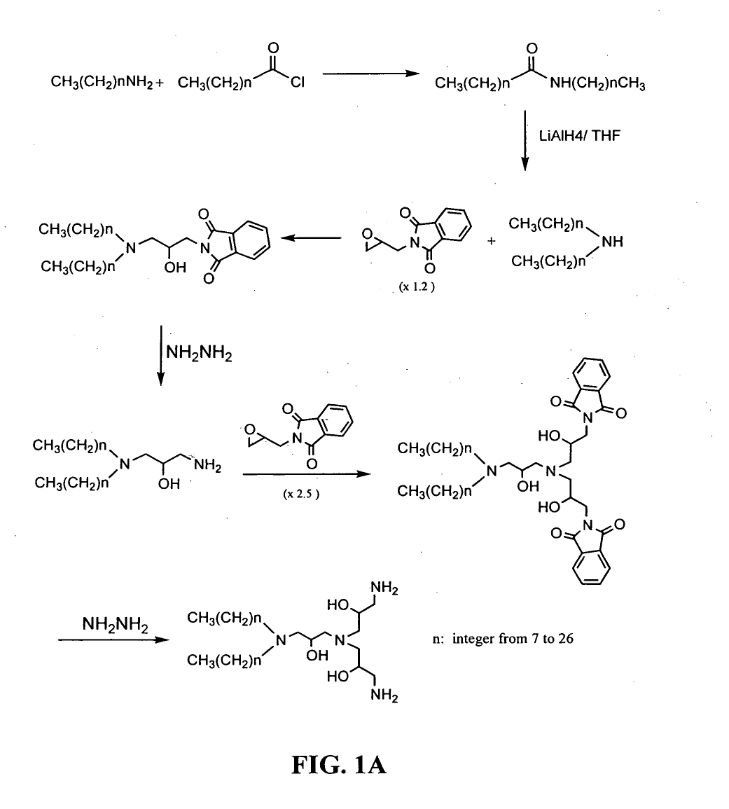

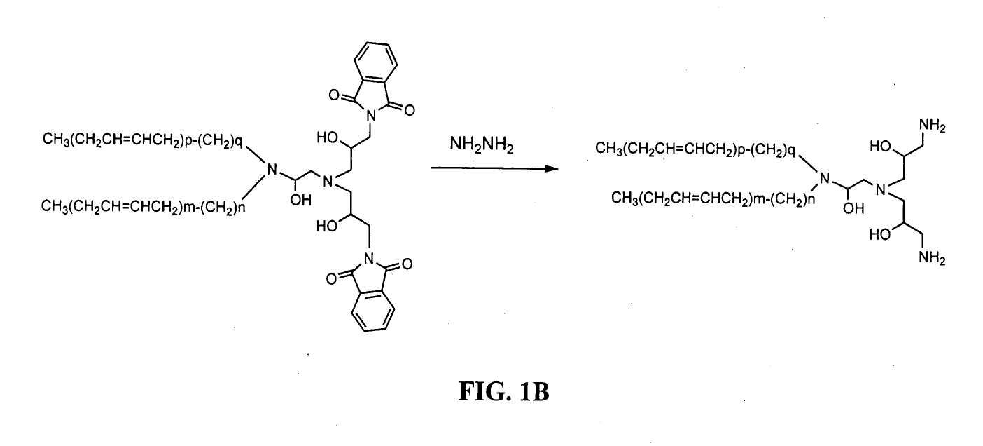

In a first class of lipids according to the invention, Q₁ is —N(R)₂ wherein R is a straight chain alkyl group having from 8 to 27 carbon atoms. The synthesis of lipids of this type is illustrated in FIGS. 1-8.

FIG. 1A shows a method of synthesizing a lipid precursor according to the invention wherein Q₁ is N(R)₂ and wherein both R substituents on the Q₁ nitrogen atom are n-alkyl substituents. In a first step of the synthesis, an n-alkyl amine is reacted with an n-alkyl carboxylic acid chloride to form an amide. In a second step of the synthesis, the amide is reduced with LiAlH₄ to form a secondary amine (i.e., a di-n-alkyl substituted amine). The di-n-alkyl substituted amine is then reacted in a third step with N-(2,3-Epoxypropyl)phthalimide to form a phthalimide adduct. This reaction product is then reacted with hydrazine to cleave the pthalimide and form the corresponding primary amine in a fourth step. In a fifth step of the synthesis, the amine is reacted with N-(2,3-Epoxypropyl)phthalimide to form a di-phthalimide adduct. Reaction of the di-phthalimide adduct with hydrazine in a final step results in cleavage of the phthalimide moieties to form the lipid precursor according to the invention.

FIG. 1B shows a step in a method of synthesizing a lipid precursor according to the invention wherein Q₁ is N(R)₂ and wherein both R substituents on the Q₁ nitrogen atom are n-alkenyl substituents. The lipid precursor of FIG. 1B can be made by a method similar to that depicted in FIG. 1A. As shown in FIG. 1B, a di-phthalimide adduct having alkenyl side chains is reacted with hydrazine to cleave the phthalimide moieties to form the lipid precursor. In FIG. 1B, m, n, p and q can be the same or different and are represented by 0 or a positive integer. The alkenyl side chains depicted in FIG. 1B are merely representative and other alkenyl side chains can also be used according to the invention.

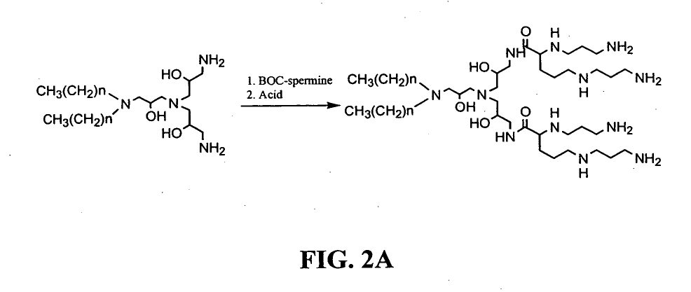

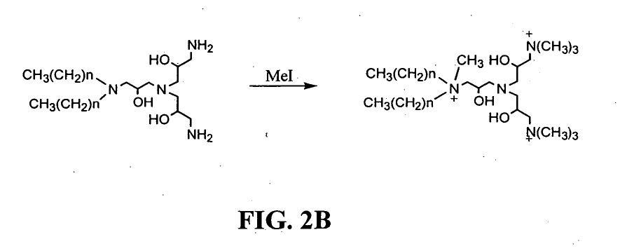

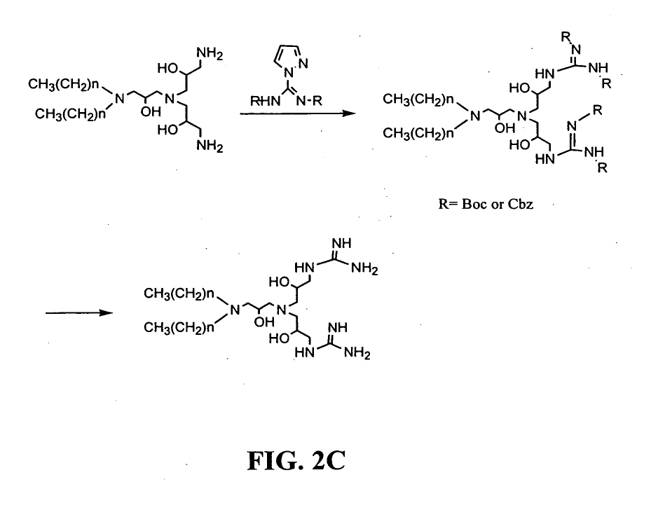

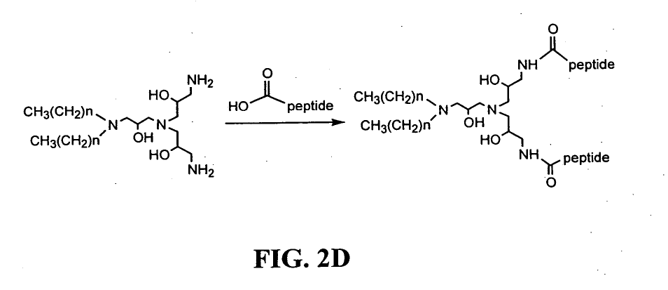

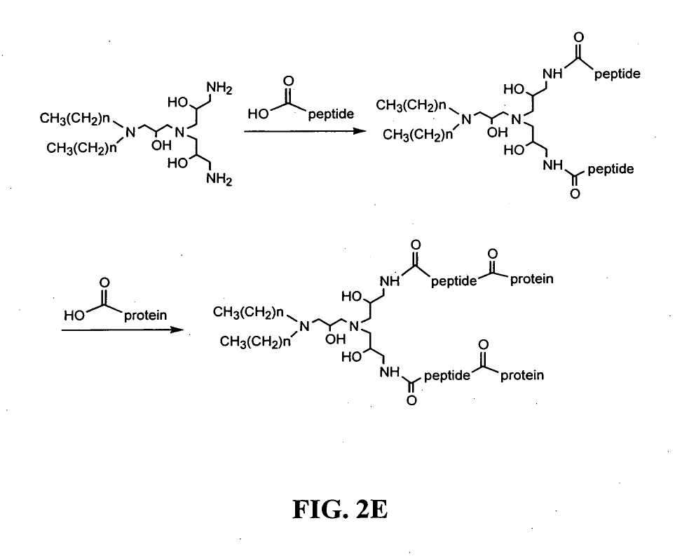

The lipid precursors as set forth in FIGS. 1A and 1B can be used to synthesize various lipids according to the invention. FIGS. 2A-2E, for example, show methods of synthesizing lipids according to the invention from the lipid precursor of FIG. 1A. In FIG. 2A, for example, the lipid precursor of FIG. 1A is reacted with BOC-spermine (spermine having the amino groups protected with t-butoxy carbonyl groups) under acidic conditions to yield an embodiment of a lipid according to the invention. In FIG. 2B, the lipid precursor of FIG. 1A is reacted with MeI (iodomethane) to yield a cationic lipid according to another embodiment of the invention.

In FIG. 2C, the lipid precursor of FIG. 1A is reacted with an N-substituted pyrazine compound to yield a lipid according to the invention. The N-substituted pyrazine compound has two amino groups both of which are substituted by a protecting group. Suitable protecting groups include BOC (t-butyloxycarbonyl) or Cbz (carbobenzyloxy) protecting groups.

In FIG. 2D, amino groups on the lipid precursor of FIG. 1A are reacted with a carboxylic acid group on a polypeptide to yield a lipid containing a polypeptide residue according to a further embodiment of the invention. The polypeptide can be a T-shaped or a linear polypeptide from either natural or non-natural amino acids. According to a preferred embodiment of the invention, the polypeptide comprises from 1 to 40 units. The polypeptides used according to the invention can be positively charged DNA condensing peptides or membrane disrupting peptides. Non-limiting examples of suitable polypeptides include polylysine, polyhistine, polyarginine, nucleus localization sequence or combinations thereof.

In FIG. 2E, amino groups on the peptide residue of the lipid of FIG. 2D are reacted with a carboxylic acid group of a protein to form a lipid containing a protein residue according to another embodiment of the invention. The protein can be a DNA condensing protein such as histone or protamine.

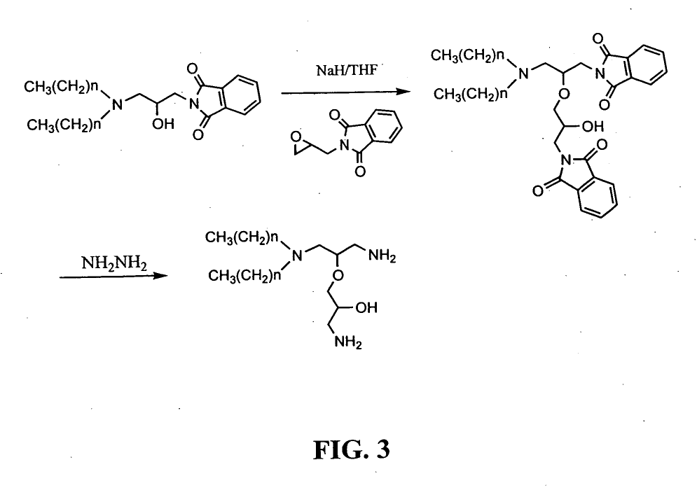

FIG. 3 shows a method of synthesizing a lipid precursor according to a further embodiment of the invention. The lipid precursor of FIG. 3 can be synthesized from the phtalimide adduct intermediate product of the synthesis depicted in FIG. 1. As can be seen from FIG. 3, the phtalimide adduct can be reacted with N-(2,3-Epoxypropyl)phthalimide under conditions in which the hydroxy group on the phthalimide adduct reacts with the epoxide group on the N-(2,3-Epoxypropyl)phthalimide to form a di-phthalimide adduct. Reaction of the di-phthalimide adduct with hydrazine in a final step results in cleavage of the phthalimide moieties to form the lipid precursor according to the invention.

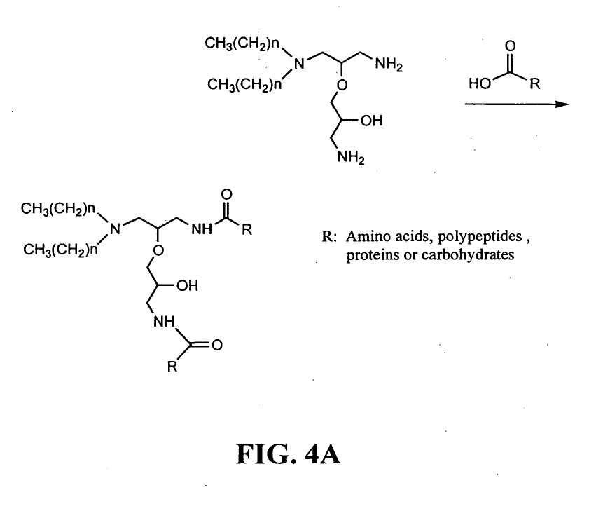

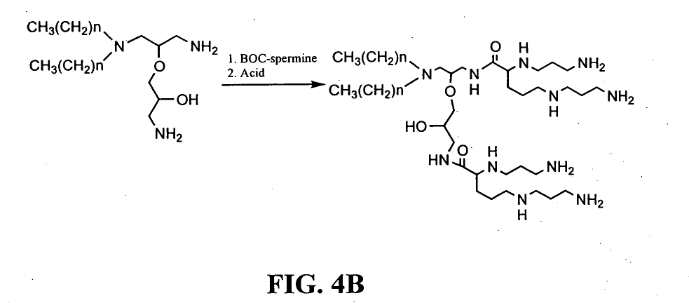

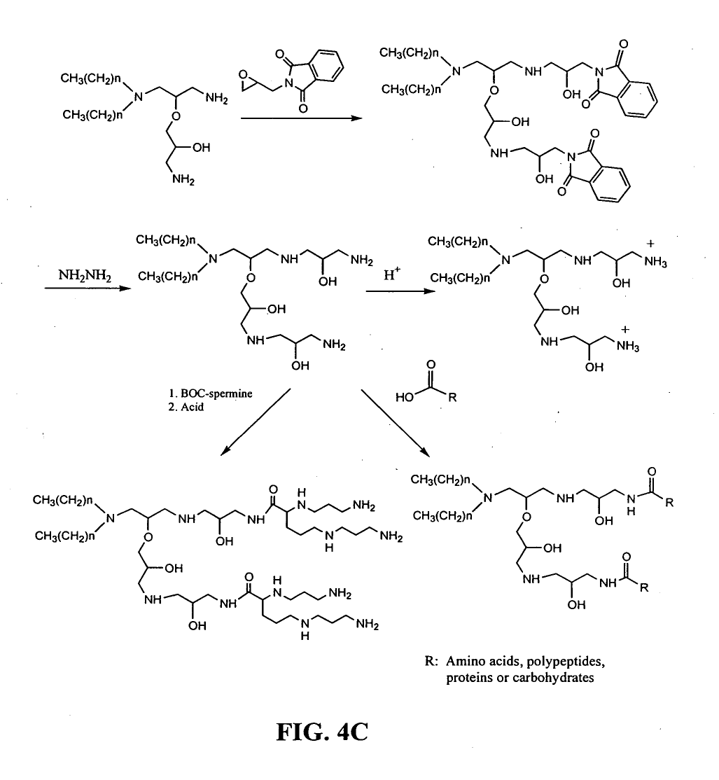

The lipid precursor of FIG. 3 can also be used to synthesize various lipids according to the invention. Examples of lipids synthesized from the precursor of FIG. 3 are shown, for example, in FIGS. 4A-4C. As shown in FIG. 4A, amino groups on the lipid precursor can be reacted with a carboxylic acid group on an amino acid, a polypeptide, a protein or a carbohydrate to obtain a lipid according to an embodiment of the invention. As shown in FIG. 4B, the lipid precursor of FIG. 3 can be reacted with BOC-spermine under acidic conditions to yield a lipid according to a further embodiment of the invention.

FIG. 4C illustrates the synthesis of higher order (i.e., wherein n≧1) lipids from the lipid presursor of FIG. 3. In a first step of the synthesis depicted in FIG. 4C, the lipid precursor of FIG. 3 is shown reacted with 2 equivalents of N-(2,3-Epoxypropyl)phthalimide. The di-phthalimide adduct reaction product is then reacted with hydrazine in a second step of the synthesis to cleave the pthalimide groups to form an intermediate product having primary amino groups. In a third step of the synthesis, the primary amino groups of the intermediate product can be reacted with BOC-spermine to form a lipid according to an embodiment of the invention. This step is shown in FIG. 4C. Alternatively, as also shown in FIG. 4C, the primary amino groups of the intermediate product can be reacted with a carboxylic acid group of an amino acid, a polypeptide, a protein or a carbohydrate to form another embodiment of a lipid according to the invention. In a further embodiment of the invention, the primary amino groups of the intermediate product can be protonated to form a cationic lipid which is also shown in FIG. 4C.

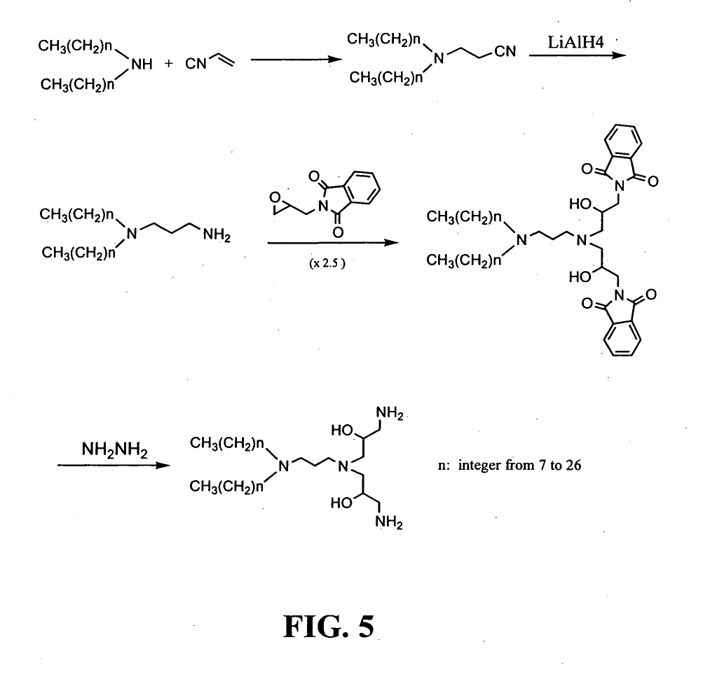

FIG. 5 shows a method of synthesizing a lipid precursor according to a third embodiment of the invention wherein, in formula I, n is 0, Q₃ is hydrogen and wherein Q₁ is N(R)₂ wherein both R substituents on the Q₁ nitrogen atom are straight chain n-alkyl groups. According to a preferred embodiment of the invention, these n-alkyl groups have from 8 to 27 carbon atoms.

The lipid precursor of FIG. 5 can be synthesized using the di-n-alkyl substituted amine intermediate product of FIG. 1. In a first step of the synthesis depicted in FIG. 5, the di-n-alkyl substituted amine intermediate product of FIG. 1 is reacted with acrylonitrile. The nitrile group on the reaction product is then reduced with LiAlH₄ to form the corresponding primary amino group which is reacted in a third step with N-(2,3-Epoxypropyl)phthalimide to form the di-phthalimide-N-substituted adduct shown in FIG. 5. Reaction of the di-phthalimide-N-substituted adduct with hydrazine results in cleavage of the phthalimide groups to form the lipid presursor according to the invention.

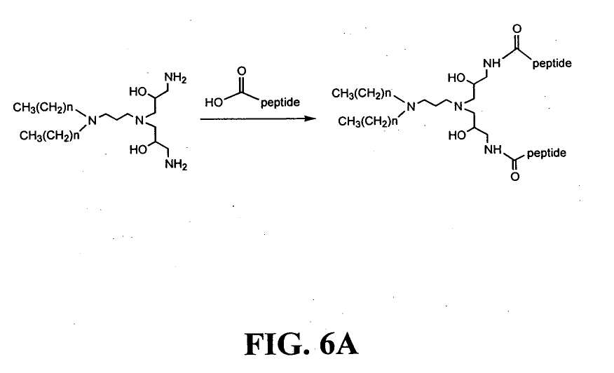

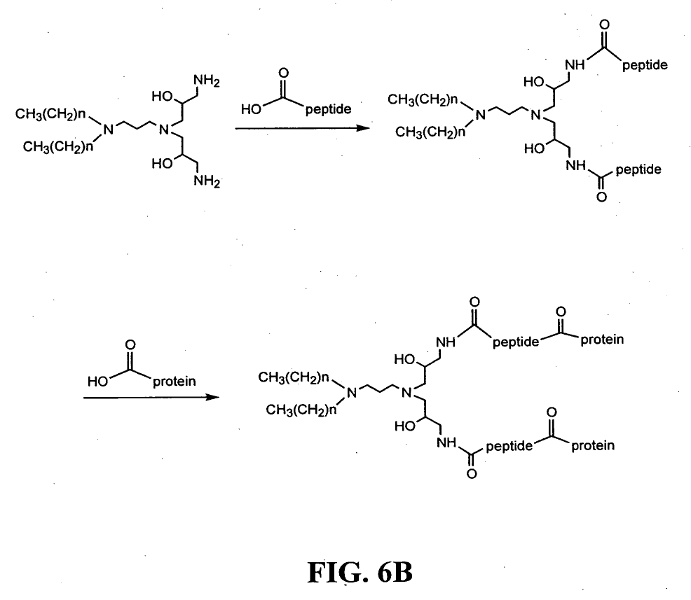

FIGS. 6A and 6B show methods of synthesizing lipids according to the invention from the lipid precursor of FIG. 5. In FIG. 6A, amino groups on the lipid precursor of FIG. 5 are reacted with carboxylic acid groups on a polypeptide to yield a lipid according to an embodiment of the invention. The polypeptide can be a T-shaped or a linear polypeptide from either natural or non-natural amino acids. According to a further preferred embodiment of the invention, the polypeptide can comprise from 1 to 40 peptide units. Polypeptides according to the invention can be positively charged DNA condensing polypeptides or membrane disrupting polypeptides. Non-limiting examples of suitable polypeptides include polylysine, polyhistine, polyarginine, nucleus localization sequence or a combination thereof.

In FIG. 6B, amino groups on each of the polypeptide residues of the lipid of FIG. 6A are reacted with the carboxylic acid group of a protein to form a lipid according to a further embodiment of the invention. According to a preferred embodiment of the invention, the protein can be a DNA condensing protein such as histone or protomine.

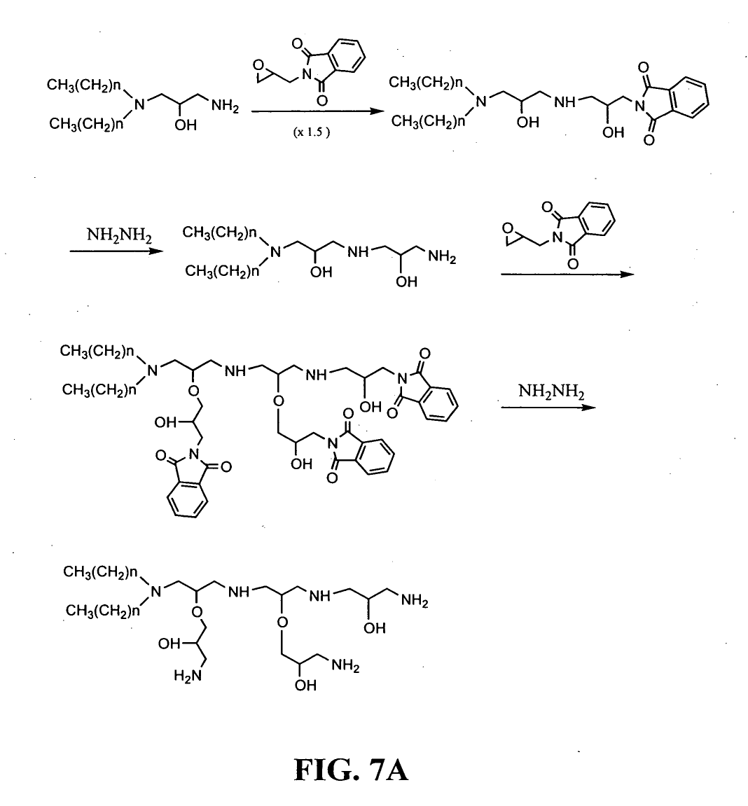

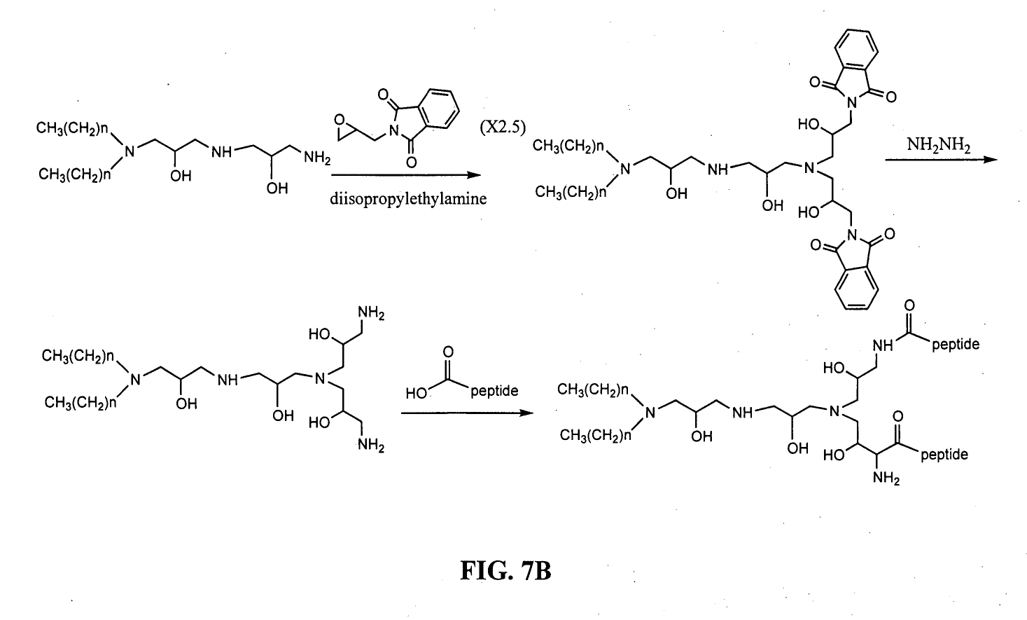

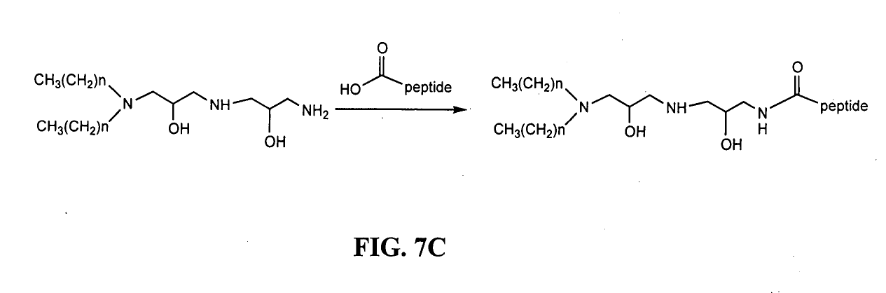

FIG. 7A shows a method of synthesizing a lipid precursor according to a further embodiment of the invention. The lipid of FIG. 7A can be synthesized using an intermediate product of the synthesis depicted in FIG. 1. As shown in FIG. 7A, the intermediate product from step 4 of the synthesis of FIG. 1 is reacted with N-(2,3-Epoxypropyl)phthalimide in a first step to form a phthalimide adduct. The phtalimide group is then cleaved from the adduct in a second step. In a third step, the reaction product of the second step is reacted with 3 equivalents of N-(2,3-Epoxypropyl)phthalimide to form a tri-phthalimide adduct. Cleavage of the phtalimide groups of the tri-phthalimide adduct with hydrazine results in the formation of the lipid precursor according to the invention.

The primary amino groups on the lipid precursor of FIG. 7A can be reacted with carboxylic acid groups on amino acids, polypeptides, proteins or carbohydrates to form lipids according to the invention. The primary amino groups can also be protonated to form a cationic lipid or reacted with N-protected spermine. These methods are discussed above with respect to the synthesis of FIG. 5.

FIG. 7B shows a method of synthesizing a lipid according to a further embodiment of the invention. The lipid of FIG. 7B can be synthesized using an intermediate product of the synthesis depicted in FIG. 7A. As shown in FIG. 7B, the intermediate product from step 2 of the synthesis of FIG. 7A is reacted with N-(2,3-Epoxypropyl)phthalimide and diisopropylethylamine in a first step. The resulting di-phthalimide adduct is then reacted with hydrazine to cleave the phthalimide moieties. As shown in FIG. 7B, each of the resulting primary amino groups can then be reacted with a carboxylic acid group on a polypeptide to form a lipid according to the invention. Although a polypeptide is shown in FIG. 7B, the primary amino groups can also be reacted with a carboxylic acid group on an amino acid, a protein or a carbohydrate to form lipids according to the invention.

FIG. 7C shows a method of synthesizing a different lipid using an intermediate product of the synthesis depicted in FIG. 7A according to a further embodiment of the invention. As shown in FIG. 7C, the amino group on the intermediate product from step 2 of the synthesis of FIG. 7A is reacted with a carboxylic acid group on a polypeptide to form the lipid. Although a polypeptide is shown in FIG. 7C, the primary amino group can also be reacted with a carboxylic acid group on an amino acid, a protein or a carbohydrate to form other lipids according to the invention.

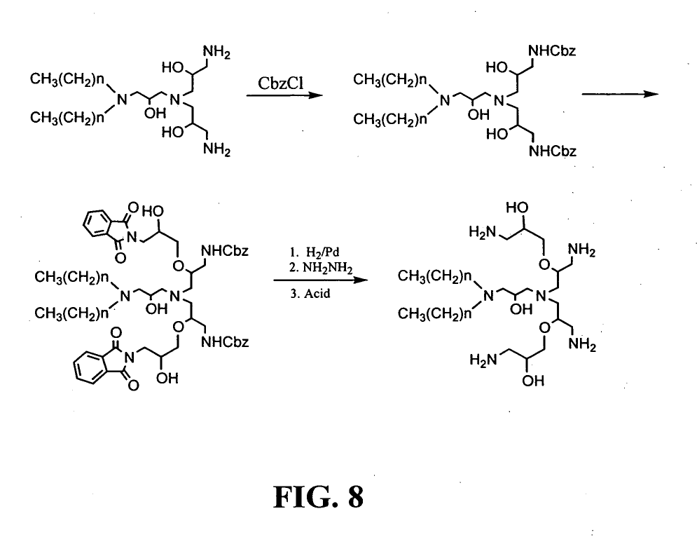

FIG. 8 shows a method of synthesizing a lipid precursor according to a further embodiment of the invention. The lipid precursor of FIG. 8 can be synthesized using the lipid precursor of FIG. 1. In a first step of the synthesis depicted in FIG. 8, the primary amino groups on the lipid precursor of FIG. 1 are protected by reaction with carboxybenzyloxy chloride. The N-protected reaction product is then reacted with N-(2,3-Epoxypropyl)phthalimide in a second step under conditions in which the hydroxy groups on the reaction product react with the epoxide groups of the N-(2,3-Epoxypropyl)phthalimide. Deprotection of the amino groups and cleavage of the phthalimide groups with hydrazine are conducted in a final step of the synthesis of the lipid precursor according to the invention.

The primary amino groups on the lipid precursor of FIG. 8 can also be reacted with carboxylic acid groups on amino acids, polypeptides, proteins or carbohydrates to form lipid compounds according to the invention. The primary amino groups can also be protonated to form a cationic lipid compound or reacted with N-protected spermine. These methods are discussed in FIG. 5 above.

In a second class of lipids according to the invention, Q₃ is O(R′), NH(R′) or S(R′), Q₄ is N(R′)₂ wherein one R′ substituent on the Q₄ nitrogen atom is represented by formula II wherein Q₆ is OR′ and the remaining R′ substituent on the Q₄ nitrogen atom is represented by the moiety of formula III wherein Q₈ is OR′.

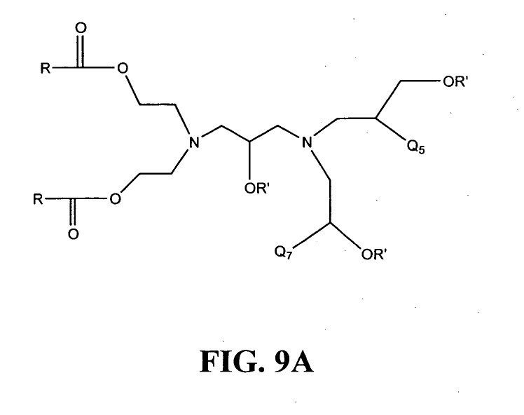

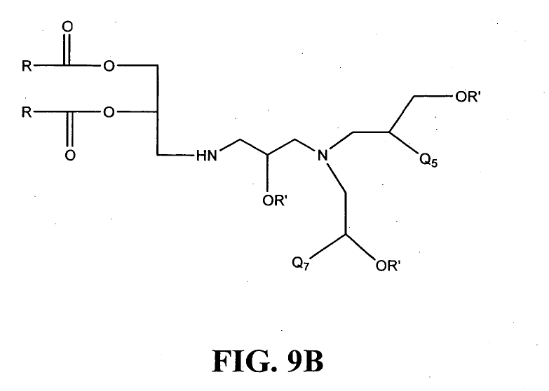

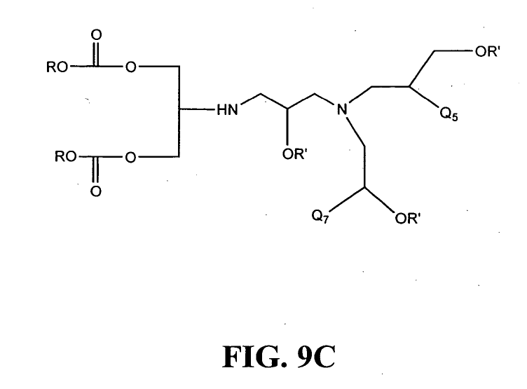

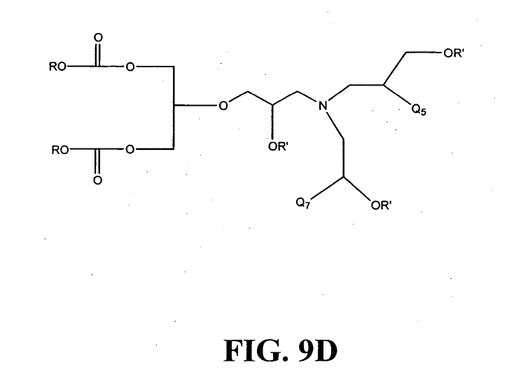

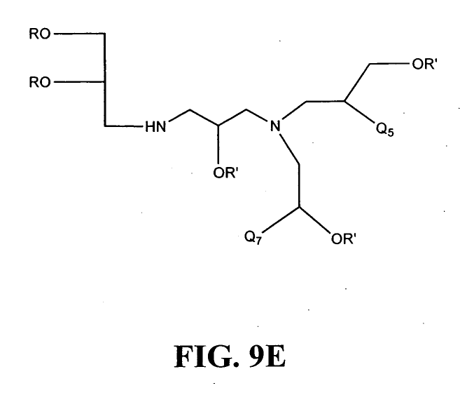

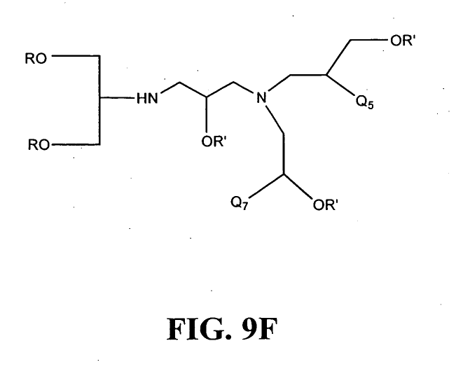

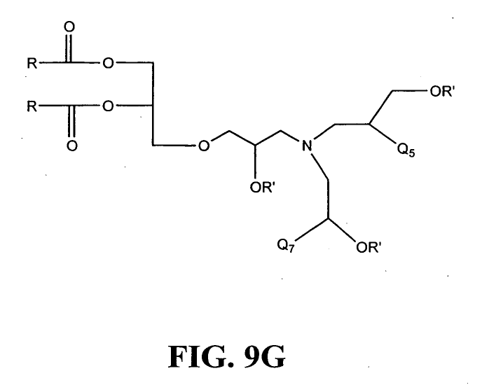

Examples of lipids of the above type where n=0, Q₁ is —N(R)₂ and Q₃ is —OR′ are represented by the general formula IV below. 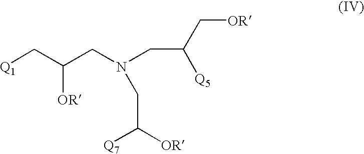
Examples of compounds corresponding to general formula IV above are listed in FIGS. 9A-9G.

In FIG. 9A, Q₁ is N(R)₂ and each of the R substituents on the Q₁ nitrogen are straight chain alkyl esters. In FIG. 9B, Q₁ is N(R)₂ and one of the R substituents on the Q₁ nitrogen atom is a branched chain alkyl ester and the remaining R substituent on the Q₁ nitrogen atom is hydrogen. In FIG. 9C, Q₁ is N(R)₂ and one of the R substituents on the Q₁ nitrogen atom is a branched chain alkyl carbonyldioxy and the remaining R substituent on the Q₁ nitrogen atom is hydrogen.

In FIGS. 9D and 9G, Q₁ is OR wherin the R substituent on the Q₁ oxygen atom is a branched chain alkyl ester. In FIGS. 9E and 9F, Q₁ is N(R)₂ wherein one of the R substituents on the Q₁ nitrogen is a branched alkyl ether and the remaining R substituent on the Q₁ nitrogen is hydrogen.

In a third class of lipids according to the invention, Q₃ is OR′, NHR′ or SR′, and Q₄ is N(R′)₂ wherein one R′ substituent on the Q₄ nitrogen atom is represented by formula II wherein Q₅ is OR and the remaining R′ substituent on the Q₄ nitrogen atom is also represented by formula II wherein Q₄ is OR.

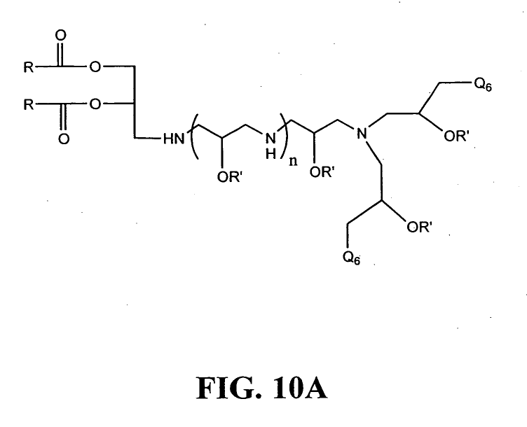

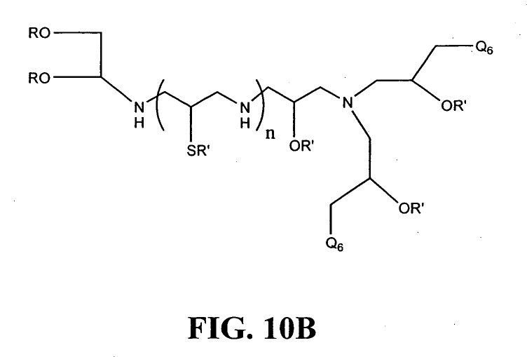

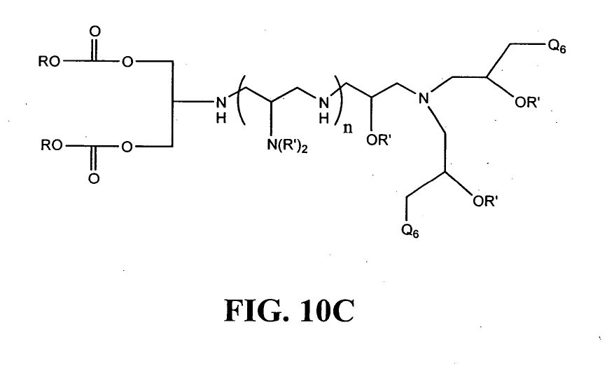

An example of a lipid of the above type wherein Q₂ is OR′ and Q₃ is OR′ is shown in FIG. 10A. In FIG. 10A, Q₁ is N(R)₂ wherein one of the R substituents on the Q₁ nitrogen is a branched alkyl ester and the remaining R substituent on the Q₁ nitrogen is hydrogen. An example of a lipid of the above type wherein Q₂is SR′ and Q₃ is OR′ is shown in FIG. 10B. In FIG. 10B, Q₁ is N(R)₂ wherein one of the R substituents on the Q₁ nitrogen is a branched alkyl ether and the remaining R substituent is hydrogen. An example of a lipid of the above type wherein Q₂ is N(R′)₂ and Q₃ is OR′ is given in FIG. 10C. In FIG. 10C, Q₁ is N(R)₂ wherein one of the R substituents on the Q₁ nitrogen is a branched alkyl carbonyldioxy and the remaining R substituent is hydrogen. In FIGS. 10A-10C, n is 0 or a positive integer. According to a preferred embodiment of the invention, n in FIGS. 10A-10C is 0-80.

In a fourth class of lipids according to the invention, Q₃ is OR′, NHR′ or SR′, Q₄ is N(R′)₂ wherein one of the R′ substituents on the Q₄ nitrogen is the moiety of formula II wherein Q₅ is OR′, and the remaining R′ substituent on the Q₄ nitrogen the moiety of formula III wherein Q₈ is OR.

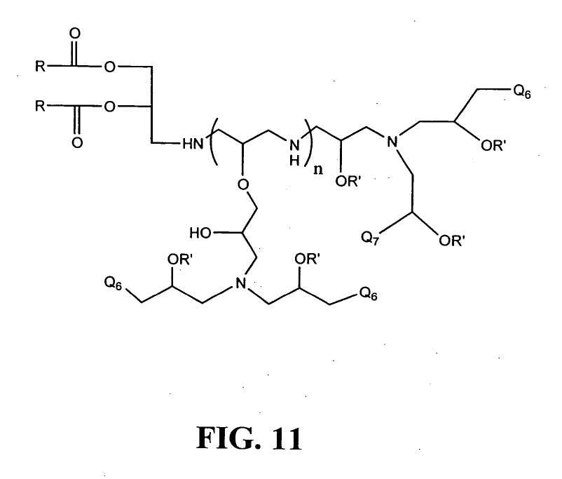

An example of a lipid of the above type wherein Q₃ is OR′ and Q₂ is —OR′ is given in FIG. 11. In FIG. 11, the R′ moiety on the Q₂ oxygen atom is the moiety of formula II wherein Q₅ is OH and Q₆ is N(R′)₂ wherein each of the R′ moieties on the Q₆ nitrogen atom are represented by formula II wherein Q₅ is OR′. In FIG. 11, n is 0 or a positive integer. According to a preferred embodiment of the invention, n in FIG. 11 is 0-80.

According to a further embodiment of the invention, one or more of the R′ substituents in the structures depicted in FIGS. 9-11 are polypeptide residues resulting from the reaction of hydroxyl groups on the lipid precursor with an amino group on a polypeptide or protein. The polypeptide according to the invention can be a T-shaped or a linear polypeptide from either natural or non-natural amino acids. According to a preferred embodiment of the invention, the polypeptide comprises from 1 to 40 units. The polypeptides or proteins according to the invention can be positively charged DNA condensing or membrane disrupting peptides or proteins. Non-limiting examples of suitable polypeptides include polylysine, polyhistine, polyarginine, nucleus localization sequences or combinations thereof.

The lipids according to the invention can be used to form lipid aggregates (i.e., liposomes) which can be used as transfection agents for the delivery of compounds into cells. Compounds that can be transfected using compounds according to the invention include DNA, RNA, oligonucleotides, peptides, proteins, carbohydrates and drugs. Methods of transfection and delivery of these and other compounds are well-known in the art.

The lipid aggregates according to the invention can be formed using a lipid aggregate forming compound such as DOPE, DOPC or cholesterol. Compounds according to the invention may also be mixed with other substances such as proteins, peptides and growth factors to enhance cell targeting, uptake, internalization, nuclear targeting and expression.

The lipids according to the invention may also be provided in a kit comprising the lipid and at least one additional component. The additional component can be one or more cells, a cell culture media, a nucleic acid, or a transfection enhancer.

According to a preferred embodiment of the invention, the transfection enhancer can be a biodegradable polymer such as a natural polymer, a modified natural polymer, or a synthetic polymer. Suitable biodegradable polymers include, but are not limited to, carbohydrates (e.g., linear or T-shaped carbohydrates) and polysaccharides such as amylopectin, hemi-cellulose, hyaluronic acid, amylose, dextran, chitin, cellulose, heparin and keratan sulfate. The transfection enhancer according to the invention can also be a DNA condensing protein (e.g., a histone or a protamine), a cell membrane disruption peptide or a ligand (e.g., a peptide or a carbohydrate) which specifically targets certain surface receptors on the cell being transfected. For example, the ligand can interact with surface receptors on the cell being transfected via ligand and receptor interactions. In this manner, transfection can be enhanced (e.g., via receptor mediated endocytosis).

The kit according to the invention may also comprise an inhibitor for one or more enzymes. These inhibitors can inhibit enzymes involved in DNA expression in the cell being transfected.

The compounds and compositions of the present invention yield lipid aggregates that can be used in the same processes used for other known transfection agents. For example, a liposome can be formed from lipid compounds according to the invention and the liposome can be contacted with a substance to be transfected to form a complex between the liposome and the substance. The complex can then be incubated with one or more cells. According to a preferred embodiment of the invention, the substance is a biologically active substance. According to a further preferred embodiment of the invention, the substance is DNA, RNA, an oligonucleotide, a peptide, a protein, a carbohydrate or a drug. The transfection methods according to the invention can be applied to in vitro or in vivo transfection of cells, particularly to the transfection of eukaryotic cells or tissue including animal cells, human cells, insect cells, plant cells, avian cells, fish cells, mammalian cells and the like.

The methods of the invention can also be used to generate transfected cells or tissues which express useful gene products. For example, the methods of the invention can be used to produce transgenic animals. The methods of the invention are also useful in any therapeutic method requiring the introduction of nucleic acids into cells or tissues, particularly for cancer treatment, in vivo and ex vivo gene therapy and in diagnostic methods. Methods of this type are disclosed, for example, in U.S. Pat. No. 5,589,466 which is herein incorporated by reference in its entirety.

The compounds and methods of the invention can also be employed in any transfection of cells done for research purposes. Nucleic acids that can be transfected by the methods of the invention include DNA and RNA from any source including those encoding and capable of expressing therapeutic or otherwise useful proteins in cells or tissues, those which inhibit expression of nucleic acids in cells or tissues, those which inhibit enzymatic activity or which activate enzymes, those which catalyze reactions (ribozymes) and those which function in diagnostic assays.

The compounds, compositions and methods of the invention can also be readily adapted to introduce biologically active macromolecules or substances other than nucleic acids into cells. Suitable substances include polyamines, polyamino acids, polypeptides, proteins, biotin and polysaccharides. Other useful materials such as therapeutic agents, diagnostic materials and research reagents can also be introduced into cells by the methods of the invention.

It will be readily apparent to those of ordinary skill in the art that a number of general parameters can influence the efficiency of transfection or delivery. These parameters include, for example, the lipid concentration, the concentration of compound to be delivered, the number of cells transfected, the medium employed for delivery, the length of time the cells are incubated with the lipid complex, and the relative amounts of cationic and non-cationic lipid. It may be necessary to optimize these parameters for each particular cell type. Such optimization can be routinely conducted by one of ordinary skill in the art employing the guidance provided herein and knowledge generally available to the art.

It will also be apparent to those of ordinary skill in the art that alternative methods, reagents, procedures and techniques other than those specifically detailed herein can be employed or readily adapted to produce the liposomal precursors and transfection compositions of this invention. Such alternative methods, reagents, procedures and techniques are within the spirit and scope of this invention.

From the foregoing, it will be appreciated that, although specific embodiments of the invention have been described herein for purposes of illustration, various modifications may be made without deviating from the spirit and scope of the invention.

1. A compound having a general structure represented by formula: 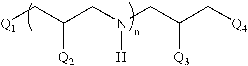
wherein:
n is 0 or a positive integer;
Q₁ is N(R)₃+, N(R)₂, O(R), or O(R)₂+ wherein each R substituent is independently selected from the group consisting of H, a straight chain or branched alkyl or alkenyl, a straight chain or branched alkyl or alkenyl ether, a straight chain or branched alkyl or alkenyl ester and a straight chain or branched alkyl or alkenyl carbonyldioxide with the proviso that at least one R substituent on the O or N atom of Q₁ is not H;
Q₃, and each Q₂ are independently selected from the group consisting of H, O(R′), N(R′)₂, NH(R″), and S(R′); and
Q₄ is selected from the group consisting of N(R′)₂, and NH(R″); wherein:
R′ is H or one the following moieties: 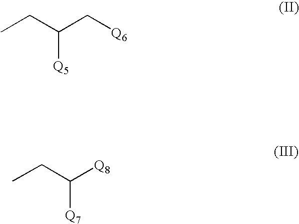
and wherein each of Q₅, Q₆, Q₇ and Q₈ are independently selected from the group consisting of N(R)₃+, N(R)₂, OR, O(R)₂+, O(R′), N(R′)₂, NH(R″), S(R), S(R)₂+ and S(R′); wherein each R substituent on Q₅, Q₆, Q₇ or Q₈is independently selected from H or a methyl group;
each R′ substituent on Q₅, Q₆, Q₇ or Q₈is as defined above for Q₄; and
each R″ substituent on Q₂, Q₃, Q₄, Q₅, Q₆, Q₇ or Q₈ is independently hydrogen or comprises a moiety selected from the group consisting of amino acid residues, polypeptide residues, protein residues, carbohydrate residues and combinations thereof.

2. The compound of claim 1, wherein Q₄is N(R′)₂ and both R′ substituents on the Q₄ nitrogen atom are represented by formula II or formula III.

3. The compound of claim 2, wherein Q₃ is H or OH.

4. The compound of claim 1, wherein Q₁ is N(R)₂ and wherein both R substitents on the Q₁ nitrogen atom are straight chain alkyl or alkenyl groups having from 8 to 27 carbon atoms.

5. The compound of claim 4, wherein Q₃ is H or OH.

6. The compound of claim 5, wherein Q₄is N(R′)₂ wherein both R′ substituents on the Q₄ nitrogen atom are represented by formula II wherein Q₅ is OH.

7. The compound of claim 6, wherein Q₆is NHR″ and wherein the R″ substituent on the Q₆ nitrogen atom comprises:
a peptide residue;
a spermine residue represented by the formula 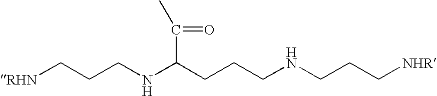
or a moiety represented by the formula:

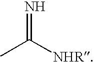

8. The compound of claim 7, wherein the R″ substituent on the Q₆ nitrogen atom comprises a peptide-protein residue.

9. The compound of claim 1, wherein Q₁ is N(R)₃+, Q₃is OH, and Q₄ is N(R′)₂ wherein both R′ substituents on the Q₄ nitrogen atom are moieties represented by formula II wherein Q₅ is OH and Q₆ is N(CH₃)₃+.

10. The compound of claim 9, wherein two of the R substituents on the Q₁ nitrogen atom are straight chain alkyl groups having from 8 to 27 carbon atoms and wherein the third R substituent on the Q₁ nitrogen atom is a methyl group.

11. The compound of claim 4, wherein Q₄is NHR″ and Q₃ is OR′ wherein the R′ substituent on the Q₃ oxygen atom is represented by formula II wherein Q₅ is OH and Q₆ is NHR′.

12. The compound of claim 11, wherein the R′ substituent on the Q₆ nitrogen atom comprises:
a spermine residue represented by the formula 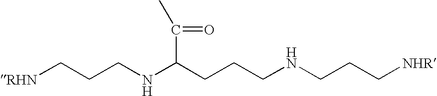
or a moiety represented by the formula:

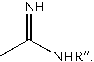

13. The compound of claim 3, wherein Q₄is N(R′)₂ wherein both R′ substituents on the Q₄ nitrogen atom are moieties represented by formula II wherein Q₅ is OH and Q₆is NHR″.

14. The compound of claim 4, wherein: Q₃ is OH; Q₄is NHR″; n=2; and each Q₂ is OR′ wherein the R′ substituent on each Q₂ oxygen atom is a moiety as represented by formula II wherein Q₅ is OH and Q₆is NHR″.

15. The compound of claim 4, wherein: n=0; Q₃ is OH; Q₄is N(R′)₂ wherein both R′ substituents on the Q₄ nitrogen atom are moieties as represented by formula II wherein Q₅ is OR′ and Q₆is NHR″; and wherein the R′ substituent on each Q₅ oxygen atom is a moiety represented by formula II wherein Q₅ is OH and Q₆is NHR″.

16. The compound of claim 1, wherein Q₃ is OR′, NHR′ or SR′ and Q₄ is N(R′)₂ wherein one R′ moiety on the Q₄ nitrogen atom is a moiety of formula II wherein Q₆ is OR′ and the remaining R′ moiety on the Q₄ nitrogen atom is represented by the moiety of formula III wherein Q₈ is OR′.

17. The compound of claim 16, wherein n=0, Q₁ is —N(R)₂ and Q₃ is OR′.

18. The compound of claim 1, wherein Q₃ is —OR′, NH(R′) or S(R′) and Q₄ is N(R′)₂ wherein both R′ substituents on Q₄ are represented by the moiety of formula II wherein Q₅ is OR′.

19. The compound of claim 18, wherein Q₃ is OR′ and wherein Q₂ is OR′, SR′, or N(R′)₂.

20. The compound of claim 1, wherein: Q₃ is OR′, NHR′ or SR′; and wherein Q₄is N(R′)₂ wherein one of the R′ substituents on the Q₄ nitrogen atom is represented by the moiety of formula II wherein Q₅ is OR′, and the remaining R′ substituent on the Q₄ nitrogen atom is represented by the moiety of formula III wherein Q₈ is OR′.

21. The compound of claim 20, wherein Q₂ and Q₃ are OR′.

22. The compound of claim 20, wherein the R′ substituent on the Q₂ oxygen atom is represented by formula II wherein Q₅ is OH and Q₆is N(R′)₂ and wherein both R′ substituents on the Q₆ nitrogen atom are represented by formula II wherein Q₅ is OR′.

23. A lipid aggregate comprising one or more molecules of a compound as set forth in claim 1.

24. The lipid aggregate of claim 23, further comprising at least one lipid aggregate forming compound.

25. A kit comprising a compound as set forth in claim 1 and at least one additional component selected from the group consisting of one or more cells, a cell culture media, a nucleic acid, a transfection enhancer and combinations thereof.

26. The kit of claim 25, wherein the kit comprises a transfection enhancer selected from the group consisting biodegradable polymers, cell membrane disruption peptides, cell surface receptor ligands, and DNA condensing proteins.

27. The kit of claim 26, wherein the transfection enhancer is a biodegradable polymer selected from the group consisting of natural polymers, modified natural polymers, synthetic polymers, carbohydrates, and polysaccharides.

28. The kit of claim 27, wherein the transfection enhancer is a polysaccharide selected from the group consisting of amylopectin, hemi-cellulose, hyaluronic acid, amylose, dextran, chitin, cellulose, heparin and keratan sulfate.

29. The kit of claim 26, wherein the transfection enhancer is a DNA condensing protein selected from the group consisting of histones and protamines.

30. The kit of claim 25, wherein the kit comprises:
a cell comprising one or more enzymes involved in DNA expression; and
an inhibitor which inhibits at least one of the one or more enzymes involved in DNA expression.

31. The kit of claim 25, wherein the kit comprises:
a cell comprising one or more surface receptors; and
a ligand which interacts with at least one of the one or more surface receptors.

32. The kit of claim 31, wherein the ligand is a polypeptide or a carbohydrate.

33. A method for introducing a biologically active substance into cells comprising:
forming a liposome from a compound as set forth in claim 1;
contacting the liposome with the biologically active substance to form a complex between the liposome and the substance; and
incubating the complex with one or more cells;
wherein the biologically active substance is not a nucleic acid.

34. The method of claim 33, wherein the substance is selected from the group consisting of a polyamine, a polyamino acid, a polysaccharide and a carbohydrate.

35. The method of claim 33, wherein the substance is a polypeptide or a protein.

36. (canceled)
